(19) United States
(12) Patent Application Publication
(10) Pub. No.: US 2025/0018083 A1
(43) Pub. Date: Jan. 16, 2025

(54) NOVEL WOUND DRESSING BASED ON CELLULOSE ACETATE FILMS CONTAINING BINARY METAL OXIDE NANOHYBRIDS

(71) Applicant: KING FAISAL UNIVERSITY, Al-Ahsa (SA)

(72) Inventors: Hany Mohamed Abd El-Lateef AHMED, Al-Ahsa (SA); Mohamed GOUDA, Al-Ahsa (SA); Mai Mustafa Khalaf ALI, Al-Ahsa (SA)

(21) Appl. No.: 18/526,914

(22) Filed: Dec. 1, 2023

Related U.S. Application Data
(62) Division of application No. 18/221,957, filed on Jul. 14, 2023.

(51) Int. Cl. A61L 15/18; A61L 15/28; A61L 15/42

(52) U.S. Cl. CPC A61L 15/18; A61L 15/28; A61L 15/42; A61L 2300/104; A61L 2300/60; A61L 2400/12

(57) ABSTRACT

Cellulose acetate, silver-vanadate, and/or gadolinium trioxide wound dressing nano-films. In particular, wound dressings comprising a cellulose acetate film comprising cellulose acetate and silver vanadate and/or gadolinium trioxide nanoparticles within a cellulose acetate matrix. Different weights of these substances can be chosen during manufacture to achieve a certain morphological appearance. These multifunctional wound dressing films could be a promising and potential option for wound healing.

### BACKGROUND

### 1. Field

The present disclosure relates to wound dressings, and in particular to wound dressing based on cellulose acetate films containing binary metal oxide nanohybrids.

### 2. Description of the Related Art

Numerous injuries, and particularly burns, require the application of some type of pad, gauze, cloth, dressing or similar covering (herein collectively called a “dressing”) to protect the wound while it is healing. Wounds, especially burns, sometimes have difficulty in healing and are frequently prone to infection because natural protective skin barriers are disrupted and are slow in repairing themselves. The most commonly used dressing material has been cotton because it is both inexpensive and readily available. However, as those who have used cotton dressings are aware, they tend to stick to the injured area, even when the surface of the area is covered with a lubricant such as petroleum jelly (“petrolatum”) or similar substance, or a medicinal agent which contains a lubricant.

Developments in the medical arts have resulted in some improvements in medical dressings, two of which are represented by Johnson & Johnson's ADAPCTIC™ brand non-adherent dressings and the Curity® brand TELFA® sterile pads sold by Kendall-Futuro Company. The ADAPTIC™ brand dressing was found to consist of a cellulose acetate pad which has been soaked in petrolatum or similar substance to impart anti-adhesion properties. While the petrolatum reduces adhesion to a wound, it's use results in a pad that is greasy and messy to handle compared to a pad without petrolatum. The TELFA® dressing consists of a polyethylene terephthalate (PET) shell and a caustic washed cotton insert, the anti-adhesion properties being imparted by the PET shell.

Current dressings, while having various qualities which may reduce adhesion or provide other benefits, typically incorporate lubricants and/or fluids or fluid releasing agents and use fibers other than cellulose acetate to provide non-adhesion properties.

While certain current dressings represent improvements in reducing dressing adhesion to wounds, further improvements in the field are highly desirous. In particular, an improved dressing of cellulose acetate would be useful in the medical arts because cellulose acetate is both chemotactic for attracting white blood cells and hydrophilic. The white cell chemotactic property of cellulose acetate fibers is desirable in a wound dressing because white blood cells aid in fighting infection. The hydrophilic property is desirable because it aids in removing excess fluids which may ooze from the surface of a wound during the healing process.

Thus, new wound dressings solving the aforementioned problems are desired.

### SUMMARY

The present subject matter relates to cellulose acetate, silver-vanadate, and/or gadolinium trioxide wound dressing nano-films. In particular, the present wound dressings comprise a cellulose acetate film comprising cellulose acetate and silver vanadate and/or gadolinium trioxide nanoparticles within a cellulose acetate matrix. Different weights of these substances can be chosen during manufacture to achieve a certain morphological appearance. These multifunctional wound dressing films could be a promising and potential option for wound healing.

In an embodiment, the present subject matter relates to a wound dressing comprising a cellulose acetate film comprising: cellulose acetate; and nanoparticles selected from the group consisting of silver vanadate nanoparticles, gadolinium trioxide nanoparticles, and combinations thereof.

In another embodiment, the present subject matter relates to a method for making a cellulose acetate-based film, the method comprising: dissolving cellulose acetate and nanoparticles selected from the group consisting of silver vanadate nanoparticles, gadolinium trioxide nanoparticles, and combinations thereof in acetone to obtain a solution; resting the solution in a sealed glass container at about 298 K for at least about 24 hours; stirring the solution for at least about 2 hours; placing the solution onto a glass plate; and evaporating the acetone to obtain the cellulose acetate-based film.

In a further embodiment, the present subject matter relates to a method of healing a wound in a patient comprising applying to a patient in need thereof a wound dressing as described herein at a site of the wound in the patient.

These and other features of the present subject matter will become readily apparent upon further review of the following specification.

### BRIEF DESCRIPTION OF THE DRAWINGS

FIG. 1A shows XRD spectra of a cellulose acetate (CA) film, a cellulose acetate film containing silver vanadate nanoparticles (Ag₃VO₄@CA), a cellulose acetate film containing gadolinium trioxide nanoparticles (Gd₂O₃@CA), and a cellulose acetate film containing silver vanadate nanoparticles and gadolinium trioxide nanoparticles (Ag₃VO₄/Gd₂O₃@CA).

FIG. 1B shows FTIR spectra of a cellulose acetate (CA) film, a cellulose acetate film containing silver vanadate nanoparticles (Ag₃VO₄@CA), a cellulose acetate film containing gadolinium trioxide nanoparticles (Gd₂O₃@CA), and a cellulose acetate film containing silver vanadate nanoparticles and gadolinium trioxide nanoparticles (Ag₃VO₄/Gd₂O₃@CA).

FIG. 2 shows EDX analysis of an Ag₃VO₄/Gd₂O₃@CA film.

FIGS. 3A-3C show SEM images of CA based films where FIG. 3A is Ag₃VO₄@CA, FIG. 3B is Gd₂O₃@CA, and FIG. 3C is Ag₃VO₄/Gd₂O₃@CA.

FIGS. 4A-4D show wettability of CA based films where FIG. 4A is a CA film, FIG. 4B is an Ag₃VO₄@CA film, FIG. 4C is a Gd₂O₃@CA film, and FIG. 4D is an Ag₃VO₄/Gd₂O₃@CA film.

FIG. 5 shows the Wettability of CA-based Ag₃VO₄/Gd: 03@CA.

### DETAILED DESCRIPTION OF THE PREFERRED EMBODIMENTS

The following definitions are provided for the purpose of understanding the present subject matter and for construing the appended patent claims.

### Definitions

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The term “optional” or “optionally” means that the subsequently described event or circumstance may or may not occur, and that the description includes instances where said event or circumstance occurs and instances in which it does not.

It will be understood by those skilled in the art with respect to any chemical group containing one or more substituents that such groups are not intended to introduce any substitution or substitution patterns that are sterically impractical and/or physically non-feasible.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

“Subject” as used herein refers to any animal classified as a mammal, including humans, domestic and farm animals, and zoo, sports, and pet companion animals such as household pets and other domesticated animals such as, but not limited to, cattle, sheep, ferrets, swine, horses, poultry, rabbits, goats, dogs, cats and the like.

“Patient” as used herein refers to a subject in need of treatment of a condition, disorder, or disease, such as an acute or chronic airway disorder or disease.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In an embodiment, the present subject matter relates to a wound dressing comprising a cellulose acetate film comprising: cellulose acetate; and nanoparticles selected from the group consisting of silver vanadate nanoparticles, gadolinium trioxide nanoparticles, and combinations thereof.

In one embodiment of the present wound dressings, the nanoparticles can be silver vanadate nanoparticles. In this regard, the silver vanadate nanoparticles can have a particle size of about 10 to about 25 nm, or of about 12.3 nm. In another embodiment of the present wound dressings, the nanoparticles can be gadolinium trioxide nanoparticles. In this regard, the gadolinium trioxide nanoparticles can have a particle size of about 180 to about 200 nm, or of about 191 nm. In one more embodiment of the present wound dressings, the nanoparticles can be a combination of silver vanadate nanoparticles and gadolinium trioxide nanoparticles. In this binary metal oxide embodiment, the gadolinium trioxide nanoparticles can have a particle size of about 315 nm to about 335 nm, or of about 324 nm.

In a further embodiment, the cellulose acetate film can have a homogenous spread of pores on it surface.

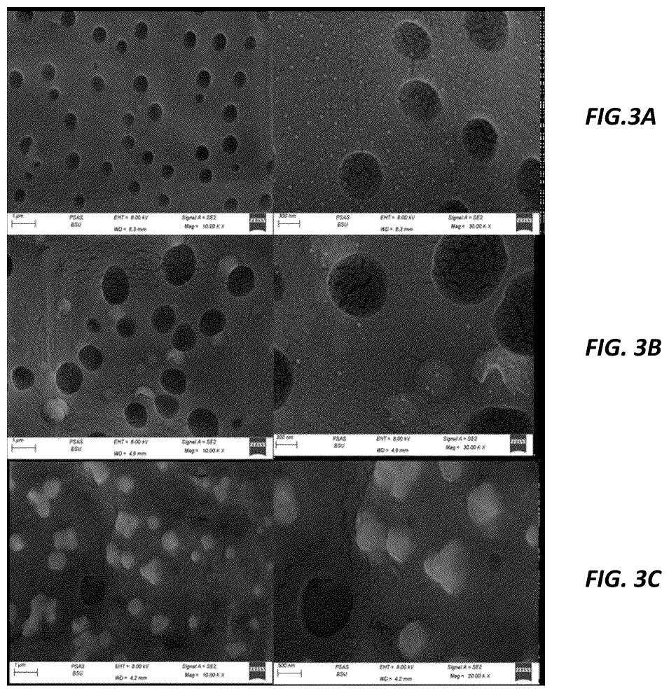

As shown in FIG. 3C, the SEM micrograph of Ag₃VO₄/Gd₂O₃@ cellulose acetate film shows diminishing pore size with growth in gadolinium trioxide grains. Porosity is the common feature in the imaged films which is essential for shaping vascularization and accelerating wound healing processes. The shown contact angle values throughout FIGS. 3A-3C signify Gd₂O₃@CA and Ag₃VO₄/Gd₂O₃@ cellulose acetate films as having the highest biocompatible potential with a contact angle of about 40.6°. The normal lung cells viability % using 1.2 μg/mL resulted in a cell viability percentage of about 102.69%, whereas using 39 g/mL resulted in a viability percentage of about 88.27%. The refractive index variation scope can be (1.88-4.62). Consequently, the present multifunctional wound dressing film could be a promising and potential option biologically, i.e., for dressing for wound healing.

In an embodiment, the silver vanadate (Ag₃VO₄) nanoparticles and/or the gadolinium trioxide (Gd₂O₃) nanoparticles can be encapsulated within the cellulose acetate. The addition of these nanoparticles may lead to an enhancement in the biological activity of cellulose acetate (CA) films.

In certain embodiments, the present wound dressings may comprise one or more further excipients, carriers, or vehicles. Non-limiting examples of suitable excipients, carriers, or vehicles useful herein include liquids such as water, saline, glycerol, polyethyleneglycol, hyaluronic acid, ethanol, and the like. Suitable excipients for nonliquid formulations are also known to those of skill in the art. A thorough discussion of therapeutically acceptable excipients and salts useful herein is available in Remington's Pharmaceutical Sciences, 18th Edition. Easton, Pa., Mack Publishing Company, 1990, the entire contents of which are incorporated by reference herein.

The present wound dressings typically contain a therapeutically effective dosage, e.g., a dosage sufficient to promote wound healing.

While human dosage levels have yet to be optimized for the present wound dressings, generally, a single wound dressing may contain from about 0.01 to 10.0 mg/kg of body weight, for example about 0.1 to 5.0 mg/kg of body weight. The precise effective amount will vary from subject to subject and will depend upon the species, age, the subject's size and health, the nature and extent of the condition being treated, recommendations of the treating physician, and the therapeutics or combination of therapeutics selected for administration. The subject may be administered as many wound dressings as is required to reduce and/or alleviate the wound.

The present wound dressings have valuable therapeutic properties, which make them commercially utilizable.

In another embodiment, the present subject matter relates to a method for making a cellulose acetate-based film, the method comprising: dissolving cellulose acetate and nanoparticles selected from the group consisting of silver vanadate nanoparticles, gadolinium trioxide nanoparticles, and combinations thereof in acetone to obtain a solution; resting the solution in a sealed glass container at about 298 K for at least about 24 hours; stirring the solution for at least about 2 hours; placing the solution onto a glass plate; and evaporating the acetone to obtain the cellulose acetate-based film.

In certain embodiments in this regard, the cellulose acetate can be dissolved in the acetone in a ratio of about 1:10 (m/v).

In a further embodiment, the present subject matter relates to a method of healing a wound in a patient comprising applying to a patient in need thereof a wound dressing as described herein at a site of the wound in the patient.

In certain embodiments in this regard, the wound dressing can include the silver vanadate nanoparticles, and vanadate from the silver vanadate nanoparticles can encourage endothelial cells to occupy collagen matrices and arrange into tubules, stimulate cell proliferation, or stimulate bone collagen formation at the site of the wound in the patient.

In another embodiment, application of the wound dressing can promote metabolite circulation and vascularization.

In one embodiment of the present methods, the wound dressing can include the gadolinium trioxide nanoparticles, which release oxonium ions (H₃O⁺) in use, thereby disrupting cellular pH and preventing development of drug resistance in the patient.

In another embodiment of the present methods, the wound dressing can include the silver vanadate nanoparticles and the gadolinium trioxide nanoparticles. According to certain of these embodiments, the wound dressing can promote wound healing through a heightened ability of the cellulose acetate film to adhere to a surface of the wound.

In the above methods, the patient is preferably a mammal, more preferably a human. Furthermore, the present wound dressing can be used in combination therapy with one or more additional therapeutic agents.

The following examples relate to various methods of manufacturing certain specific wound dressings and their use as described herein.

### EXAMPLES

### Example 1

Preparation of Silver Vanadate (Ag₃VO₄)

Sodium orthovanadate (Na₃VO₄) and silver chloride (AgCl) were obtained from PubChem. The co-precipitation method was used to prepare silver vanadate according to the following equation:

3AgCl+Na₃VO₄→3NaCl+Ag₃VO₄

To prepare Ag₃VO₄, about 5 g of AgCl was dissolved in deionized water, and 2.14 g of Na₃VO₄ was dissolved in another beaker at 343.15 K till the solution become clear. Then, the Na₃VO₄ solution was dropped wisely over the solution of AgCl and a few drops of NaCl was added to the solution to adjust the pH. After that, the precipitated powder was washed 4 times and then dried in a drier furnace. Finally, the powder was ground with a mortar, and Ag₃VO₄ nanoparticles were obtained.

### Example 2

Preparation of Cellulose Acetate Film with Various Oxide Concentrations

Silver-vanadate, gadolinium oxide, and cellulose acetate (CA) were bought from PubChem. Casting methodology helped create the CA films.

CA was dissolved in acetone at a ratio of 1:10 (m/v) with the powder (silver-vanadate and/or gadolinium oxide) components to create a film-genic solution, which then rested for 24 hours at 298 K in a sealed glass. After that, the solution was magnetically stirred for 2 hours. The mixture was put onto a glass plate and allowed to sit there until the solvent had completely evaporated. The first film was prepared from pure CA, while the other films were prepared by adding the nanoparticles to the CA solution with determined concentrations. The name of the cellulose acetate film was FAC.

### Example 3

### Qualitative and Quantitative Structural Investigation

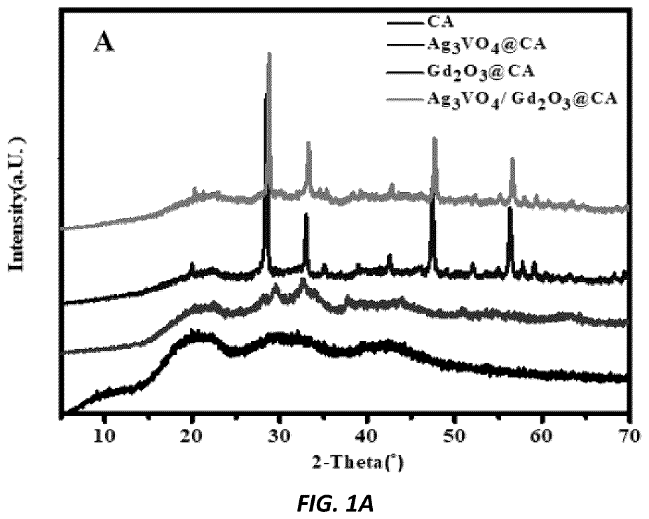

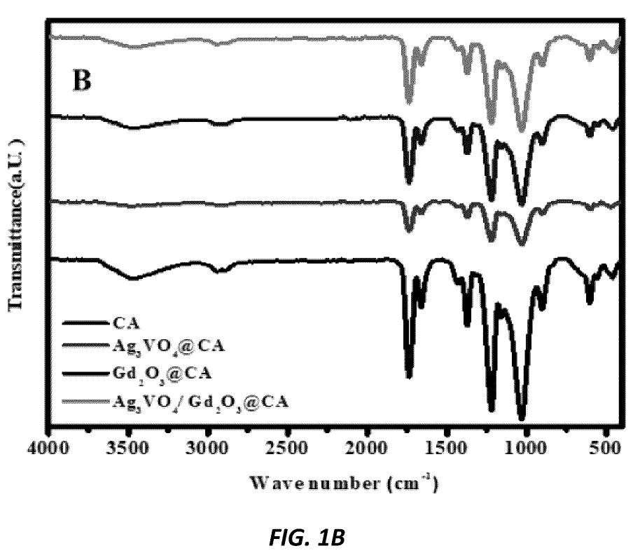

Films made of cellulose acetate were structurally investigated by XRD data. As can be seen in FIG. 1A, CA has several strong, overlapping peaks, with the main broad peak located at 2=20.5. The large peak at scope from 30° to 50° is assumed to be caused by the amorphous component of CA.

Without any proof of the existence of separated silver metal, the a and B phases are indexed to the cards (JCPDS 19-1151), and (JCPDS 29-1154), respectively. The peak of excess silver in silver vanadate-containing films is positioned at 38.4°. Peaks confirm the coexistence of silver vanadate's major phase β and minor phase α, respectively.

The primary peaks of Gd₂O₃ are seen at 28.6, 33.1, 47.3, and 56.2°, which correspond to unit cell planes (222), (400), (440), and (622). The XRD diffraction graph (FIG. 1A) confirms the presence of the investigated chemical.

In terms of FTIR spectra, as shown in FIG. 1B, signals at 1024, 1219, 1374, 1668, and 1741 cm⁻¹ corresponding to C—O—C, C—O—C, C-methyl group, C—O, and C—O, respectively, confirm the presence of CA. The weak band at 1654 cm⁻¹ and the strong band at 1743 cm⁻¹ overtone the VO terminal in silver vanadate, respectively. Peak intensity changes support the compatibility of various insertions into CA film.

As a result, FIG. 1B shows the FTIR spectra of the four films, each of which confirms its corresponding chemical structure. The Gd—O stretching maxima in several NCs are close to 535 cm⁻¹. Peaks at 914, and 836 cm⁻¹ are characteristic Ag₃VO₄ compositions. In summary, the FTIR spectra show that the chemical under investigation is present.

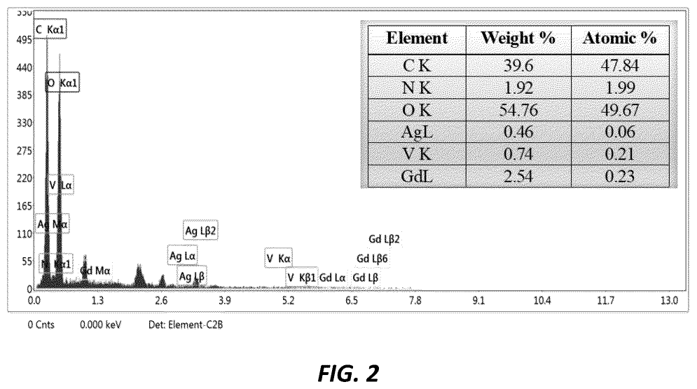

Through EDX analysis, as seen in FIG. 2, the quantitative and qualitative elemental findings of the Ag₃VO₄/Gd₂O₃@CA film are gathered. The results of the quantitative research are based on the signal intensities. The EDX data confirms the detection of C, N, O, Ag, V, and Gd with 39.6, 1.92, 54.76, 0.46, 0.74, and 2.54%. Because oxygen was linked to each element separately, it occupies without a doubt the highest atomic percentage, which appears at 0.5 keV, as shown in FIG. 2.

### Example 4

### Morphological Study

SEM micrographs as shown in FIGS. 3A-3C exhibit Ag₃VO₄@CA, Gd₂O₃@CA, and Ag₃VO₄/Gd₂O₃@CA. The Ag₃VO₄@CA porous film displays nanostructured silver vanadate as tiny dots upon the cellulose acetate matrix. The average grain size of silver vanadate is 12.3 nm. The film of Gd₂O₃@CA also shows a porous structure with wider pores size and rougher structure. Both binary structures display a homogenous spread of pores upon their surface. The Gd₂O₃ grains appeared in an average size of 191 nm. Finally, merging the three ingredients in Ag₃VO₄/Gd₂O₃@CA film diminishing in pores size with growth in gadolinium trioxide grains, that its average size reaches 324 nm. Porosity is thought to be essential for shaping vascularization and accelerating wound healing processes and the ability to transmit nutrients. Therefore, if the porosity depends on the chemical composition, it might be improved by changing the amount and kind of elements, as demonstrated in FIGS. 3A-3C.

### Example 5

### Contact Angle Properties for Cellulose Acetate Films

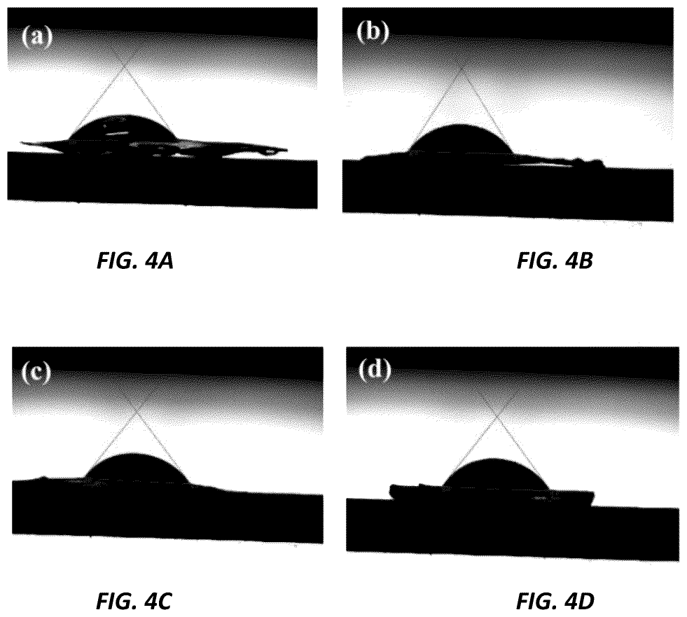

The average of contact angles for pure CA, Ag₃VO₄@CA, Gd₂O₃@CA, and Ag₃VO₄/Gd₂O₃@CA films are 41.5±0.875, 46.4±0.345, 40.6±1.035, and 40.6±0.09°, as shown in FIGS. 4A-4D. The contact angle values signify Gd₂O₃@CA and Ag₃VO₄/Gd₂O₃@CA films as the highest biocompatible potential. Additionally, the reduced contact angle provides more opportunity for adhesion. To put it briefly, surface polarity, contact angle value, and adhesion ability are all strongly impacted by chemical composition.

### Example 6

Cell Viability Percentage Upon the Utilization of Ag₃VO₄/Gd₂O₃@CA Against Normal Lung Cells

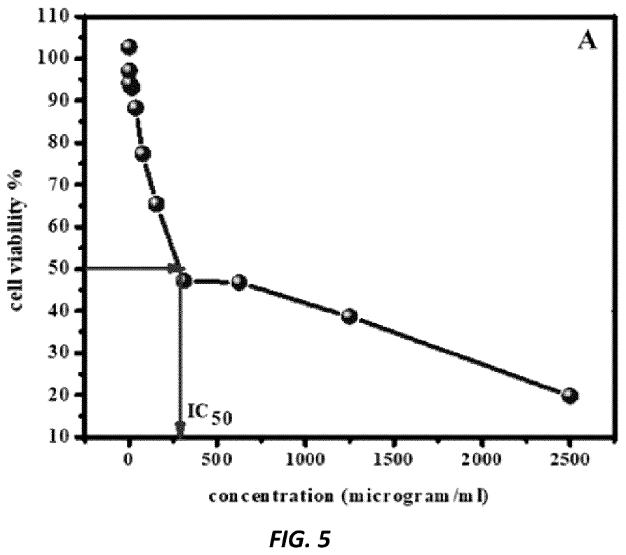

The impact of Ag₃VO₄/Gd₂O₃@CA on normal cell line viability was studied. Ag₃VO₄/Gd₂O₃@CA concentrations are plotted against normal cell lines viability in FIG. 5. When normal lung cells (A138) were tested, it was discovered that using 1.2 μg/ml resulted in a cell viability percentage of 102.69% whereas using 39 g/ml resulted in a viability percentage of 88.27%. Additionally, the use of 1250 and 2500 μg/ml significantly reduced the viability percentage of normal cells, bringing it to 38.65 and 19.81%, respectively.

According to IORDACHESCU et al. (2002), vanadate has a variety of effects that depend on the exposure level. Vanadate exhibits a mitogenic tendency at low concentrations, to a maximal level at 100 μM, acting as a growth factor. Vanadate, however, exhibits obvious decreases in cell viability and proliferation at high concentrations, suggesting that cytotoxicity is caused by the release of reactive oxygen species. Epidermal growth factor (EGF), platelet-derived growth factor (PDGF), and fibroblast growth factor (FGF) play critical roles in wound healing. Briefly, vanadate has been shown to mirror the effects of fibroblast growth factor in that it encourages endothelial cells to occupy collagen matrices and arrange into tubules. It also stimulates cell proliferation and bone collagen formation in vitro. In fact, the topological characteristics of the composition examined, such as porosity, strongly influence the interaction mechanism between the injured tissues and the implant film.

The high porosity surface promotes metabolite circulation and vascularization. The degree of roughness and wettability also have a direct impact on the biological performance of the film. Furthermore, the number, size, and distribution of the implanted silver have an impact on the efficiency of the released reactive oxy-species. Additionally, the cellular pH is disrupted due to the released oxonium ions (H₃O⁺) from Gd₂O₃ mineral trioxide, which also prevents the development of drug resistance.

In general, surface morphology, type, and quantity of composition constituents play a significant role in the potential for bio-applicability; as a result, they are the tools to modify the physical and chemical properties of the created film composite. As a result, the insertion of silver-vanadate and gadolinium trioxide promotes healing indirectly by improving the ability of the produced nano-films composition to adhere to the surface of live tissues.

It is to be understood that the wound dressings are not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1-11. (canceled)

12. A method for making a wound care cellulose acetate-based film, the method comprising:
dissolving cellulose acetate and nanoparticles selected from the group consisting of silver vanadate nanoparticles and gadolinium trioxide nanoparticles, and combinations thereof in acetone to obtain a solution;
resting the solution in a sealed glass container at about 298 K for at least 24 hours;
stirring the solution for about 2 hours;
placing the solution onto a glass plate; and
evaporating the acetone to obtain the wound care cellulose acetate-based film;
wherein no plasticizer is added to the solution.

13. The method of claim 12, wherein the cellulose acetate is dissolved in the acetone in a ratio of about 1:10 (m/v).

14. A method of healing a wound in a patient comprising applying to a patient in need thereof a wound dressing at a site of the wound in the patient; wherein the wound dressing comprises a cellulose acetate film comprising:
cellulose acetate; and
nanoparticles selected from the group consisting of silver vanadate nanoparticles and gadolinium trioxide nanoparticles, and combinations thereof.

15. The method of claim 14, wherein the wound dressing includes the silver vanadate nanoparticles, and vanadate from the silver vanadate nanoparticles encourages endothelial cells to occupy collagen matrices and arrange into tubules, stimulates cell proliferation, or stimulates bone collagen formation at the site of the wound in the patient.

16. The method of claim 14, wherein application of the wound dressing promotes metabolite circulation and vascularization.

17. The method of claim 14, wherein the wound dressing includes the gadolinium trioxide nanoparticles, which release oxonium ions in use, thereby disrupting cellular pH and preventing development of drug resistance in the patient.

18. The method of claim 14, wherein the wound dressing includes the silver vanadate nanoparticles and the gadolinium trioxide nanoparticles.

19. The method of claim 18, wherein the wound dressing promotes wound healing through a heightened ability of the cellulose acetate film to adhere to a surface of the wound.

20. The method of claim 14, wherein the nanoparticles are the silver vanadate nanoparticles.

21. The method of claim 20, wherein the silver vanadate nanoparticles have a particle size of about 10 to about 25 nm.

22. The method of claim 21, wherein the silver vanadate nanoparticles have a particle size of about 12.3 nm.

23. The method of claim 14, wherein the nanoparticles are the gadolinium trioxide nanoparticles.

24. The method of claim 23, wherein the gadolinium trioxide nanoparticles have a particle size of about 180 to about 200 nm.

25. The method of claim 24, wherein the gadolinium trioxide nanoparticles have a particle size of about 191 nm.

26. (canceled)

27. The method of claim 2618, wherein the gadolinium trioxide nanoparticles have a particle size of about 315 nm to about 335 nm.

28. The method of claim 27, wherein the gadolinium trioxide nanoparticles have a particle size of about 324 nm.

29. The method of claim 14, wherein the cellulose acetate film has a homogenous spread of pores on it surface.

30. The method of claim 17, wherein the oxonium ions are hydronium ions (H₃O⁺).
